(19) United States
(12) Patent Application Publication
(10) Pub. No.: US 2025/0193594 A1
(43) Pub. Date: Jun. 12, 2025

(54) AUDIO SYSTEM

(71) Applicant: Oliver Odysseus Schuster, Innsbruck (AT)

(72) Inventor: Oliver Odysseus Schuster, Innsbruck (AT)

(21) Appl. No.: 18/845,972

(22) PCT Filed: Feb. 13, 2023
(86) PCT No.: PCT/EP2023/053414

§ 371 Date: Sep. 11, 2024

(30) Foreign Application Priority Data
Mar. 22, 2022 (AT) A 74/2022

(51) Int. Cl. H04R 3/04

(52) U.S. Cl. CPC H04R 3/04

(57) ABSTRACT

An audio system for reproducing amplified output audio signals by at least one loudspeaker of the audio system. The audio system has a signal processing unit for actuating the loudspeaker, including a digital signal processor and at least one digital/analog converter and at least one amplifier. The signal processing unit is connected/connectable to a data source for providing input audio signals to be amplified, or the data source is integrated into the signal processing unit. The audio system has a reproduction mode and a hearing test mode and additionally a data storage device for storing a result of the at least one, frequency-dependent listening test carried out with the audio system. The loudspeaker is a multi-path loudspeaker having at least two mutually different frequency band speakers for reproducing the amplified output audio signal, with the at least two mutually different frequency band speakers each having distinct useful frequency bands.

### CROSS-REFERENCE TO RELATED APPLICATIONS

This application is a 371 National Phase of PCT/EP2023/053414, filed Feb. 13, 2023, which claims priority from Austrian Patent Application No. A74/2022, filed Mar. 22, 2022, all of which are incorporated herein by reference as if fully set forth.

### TECHNICAL FIELD

The present invention relates to an audio system for reproducing amplified output audio signals by means of at least one loudspeaker of the audio system, wherein the audio system has a signal processing unit for actuating the loudspeaker, comprising a digital signal processor and at least one digital/analog converter and at least one amplifier, wherein the signal processing unit is connected or can be connected to a data source for providing input audio signals to be amplified, or the data source is integrated into the signal processing unit, wherein the audio system has a reproduction mode for reproducing the amplified output audio signals and a hearing test mode for carrying out at least one, frequency-dependent hearing test with a listener, and additionally a data storage device for storing a result of the at least one, frequency-dependent hearing test carried out with the audio system.

### BACKGROUND

Audio systems today are known in a wide variety of configurations and for a wide variety of applications. They are also used to play back music and spoken text, as well as a variety of other audio signals. Audio systems are used both in private settings, such as in one's own home, but also for smaller and larger events and the like.

As people age, many will gradually experience a deterioration in their hearing capacity without the person immediately noticing it. The onset of hearing loss is one of the most common everyday problems. It often leads to voices and dialogs becoming increasingly poorly understood, e.g. when watching TV or listening to other audio signals. However, this deterioration does not usually occur uniformly over the entire audible frequency spectrum. Rather, it is often the case that the hearing capacity of different people changes or deteriorates in different frequency ranges in a highly individual way.

Nowadays, in the event of a corresponding deterioration in the hearing capacity, it is common to use hearing aids or the like which are individually adjusted to the person in question by the audiologist by means of frequency-dependent hearing tests. However, the individual cannot do this himself or herself, but must be assisted by the specialist.

A generic audio system, which allows the listener to perform a frequency-dependent hearing test himself or herself, in order to take this into account subsequently in the reproduction of amplified output audio signals, is known from DE 10 2006 015 497 B4, for example.

### SUMMARY

The object of the invention is then to further improve such audio systems as mentioned above, in which the listener can carry out the frequency-dependent hearing test himself or herself.

In order to achieve this object, the invention proposes, based on an audio system as mentioned above, that the loudspeaker is a multi-path loudspeaker having at least two distinct frequency band speakers for reproducing the amplified output audio signal, wherein the at least two distinct frequency band speakers have distinct frequency bands.

In contrast to the above-mentioned prior art, in which the output audio signals are reproduced over their entire frequency spectrum via one single-path loudspeaker, the invention now provides that the loudspeaker is a so-called multi-path loudspeaker, in which two or more frequency band speakers are provided which have distinct useful frequency bands. Distinct means in this case that the useful frequency bands are simply not identical. However, they may optionally overlap or also be completely separate from each other.

The reproduction of the amplified output audio signal via at least two distinct frequency band speakers, which differ in their useful frequency bands, has the advantage that the output audio signal can be reproduced over its entire frequency spectrum much better than in the prior art, in which the entire frequency spectrum of the output audio signal is reproduced via a single frequency band speaker or a single loudspeaker. In this way, the result of the frequency-dependent hearing test carried out with the audio system itself by the listener can be substantially better used to ensure that specific hearing impairments in the frequency spectrum in the at least one listener are individually compensated. Thus, the listener can independently and without the help of third parties, and in particular without a visit to the audiologist, optimally adapt the audio system according to the invention to the current actual state of their own hearing capacity, so that the output audio signals can be reproduced by the audio system in a manner that is optimally adapted to the individual or to multiple individuals.

By repeating the frequency-dependent hearing test with the audio system according to the invention from time to time, the respective person can thus also monitor the development of their own hearing capacity and detect changes in the individual hearing capacity at an early stage. The result of the frequency-dependent hearing test carried out with the audio system stored in the data storage device of the audio system can also be displayed graphically for this purpose via a corresponding screen or the like, e.g. of the audio system. Multiple frequency-dependent hearing tests can also be displayed at the same time to visualize changes and allow monitoring of the development of the individual hearing capacity. It may equally well be possible that audio systems according to the invention provide a way to manually edit the results of the frequency-dependent hearing test recorded with the audio system and stored in the data storage device. Multiple results of frequency-dependent hearing tests can also be stored in the data storage device of the audio system. These can be, for example, hearing tests from different people. It is also possible to perform one frequency-dependent hearing test with the right ear and another frequency-dependent hearing test with the left ear of a person and to store the results of these hearing tests in the data storage device. In addition to the manual or even automated post-processing of the results of such frequency-dependent hearing tests, averaging of two or more such frequency-dependent hearing tests can also be carried out. This can be used, for example, in the case of multiple persons in the audio system according to the invention, for determining and storing an averaged gain function and for reproducing amplified output audio signals. Of course, in the case of an individual person the results of frequency-dependent hearing tests with the right ear and with the left ear can also be averaged, in order to use this for the reproduction of amplified output audio signals in the audio system according to the invention.

In the frequency-dependent hearing test, the hearing capacity of the particular person or the particular ear is determined at different frequencies over the normally audible frequency range. The frequency-dependent hearing test thus reflects the hearing capacity as a function of frequency. In the frequency-dependent hearing test, the lower limit of the hearing capacity, i.e. the hearing threshold, is usually determined. For this purpose, in the hearing test, as is known per se, audio signals can be played back at certain frequencies in sequence, starting quietly and then louder and louder. As soon as the person under test or the ear under test hears the signal at the corresponding frequency and the corresponding volume, the hearing threshold at that frequency is determined. In order to be able to notify the audio system that this is the case, it is advantageously provided that for carrying out the frequency-dependent hearing test the audio system is connected or can be connected to an input unit which can be operated by the listening person, or this input unit is integrated into the audio system. The input unit can be connected to the audio system wirelessly or by wires. Of course, the input unit can also be integrated directly into the audio system. For example, it can be a simple push button connected by wires, which the person can press when they hear the signal at each frequency. It can also involve wireless remote controls or the like. However, modern communication devices such as telephones, smartphones, tablets, browser-based interfaces and the like can be equally well integrated into the audio system or cooperate with them to be used as an input unit. In such input units, which have a screen or the like themselves, for example, a menu navigation for carrying out the hearing test for the respective person or the result of the respective hearing test can be displayed on the screen of this input unit.

In addition to or instead of the frequency-dependent determination of the hearing threshold, i.e. the lower limit of the hearing capacity, the frequency-dependent hearing test can also determine the individual discomfort threshold in a frequency-dependent manner. This specifies the maximum volume at which sounds can be reproduced at a certain frequency for the person or the ear in question, so that this is not yet perceived by the individual as uncomfortable or too loud. The input units mentioned above can also be used for this purpose to perform the frequency-dependent hearing test. The result of the measurement of the discomfort threshold can be used, for example, to limit the maximum volume at which the audio system reproduces the amplified output audio signals.

Appropriate input units such as smartphones, tablets or the like can also be used to allow the audio system to automatically detect which person is currently using the audio system. Thus, the audio system can then also use a result of the frequency-dependent hearing test individually stored in the data storage device for reproducing amplified output audio signals.

The stored result of the frequency-dependent hearing test can be a frequency-dependent hearing loss curve as such, but also a frequency-dependent gain function calculated from it.

Audio systems according to the invention are used for reproducing audio signals which are amplified in accordance with the frequency-dependent hearing tests. The amplified audio signals reproduced by the audio system in reproduction mode are referred to as output audio signals. The audio system could also be referred to as an audio unit or an audio reproduction device, or similar term. The input audio signal to be amplified by the audio system is an audio signal that the audio system reads in from a data source, amplifies according to the frequency-dependent hearing test and then simply reproduces as an output audio signal. The input audio signal is thus the audio signal that is read in from the data source by the audio system.

The data source can be connected or is connected to the audio system. The type of connection can be both wireless and wired. The data source can also be directly integrated into the signal processing unit of the audio system, however. The data source is a storage device of known type and can store audio signals here as input audio signals. The data source may also be a radio, the audio output of a television set, or some other arbitrary data source, which are connected or can be connected to the audio system and from which corresponding audio signals can be downloaded or read in as input audio signals for amplification by the audio system. In any case, audio systems according to the invention have a reproduction mode in which amplified output audio signals are reproduced. In addition, audio systems according to the invention, however, also have a hearing test operating mode, which is used for performing the at least one frequency-dependent hearing test and then also for storing the test result.

As already mentioned, audio systems according to the invention have at least one so-called multi-path loudspeaker as the loudspeaker(s). Multi-path loudspeakers are loudspeakers with two or more frequency band speakers, wherein the frequency band speakers differ in their useful frequency bands. Frequency band speakers are sound generators or sound transducers that are optimized for the reproduction of audio signals in a specific limited frequency band. The useful frequency band is the frequency band in which the respective frequency band speaker can optimally reproduce sound or tones due to its design. Depending on the location of their useful frequency band, such frequency band speakers are also referred to in the prior art as e.g. tweeters, midrange speakers or woofers. A multi-path loudspeaker according to the invention can have two, three, four or even more frequency band speakers with different useful frequency bands. A minimum variant consists, for example, of a multi-path loudspeaker having a tweeter and a woofer as frequency band speakers. In preferred variants, it is provided that the multi-path loudspeaker has three frequency band speakers, namely a tweeter, a midrange speaker and a woofer. In any case, the frequency band speakers are the parts of the multi-path loudspeaker that convert corresponding electrical signals into sound or tones and thus reproduce the amplified output audio signal.

Audio systems according to the invention can be designed as so-called all-in-one systems, which, apart from, say, a corresponding connection to a data source for the input audio signals and a power connection, otherwise need no further connections to the outside for their function. In such variants, intranet or Internet connections or the like to the outside can be omitted, so that no individual data such as the results of frequency-dependent hearing tests are transferred outside the system. In addition, such systems have the advantage that they can also be operated independently of other connections. Alternatively, however, it is of course also possible to design audio systems according to the invention with corresponding interfaces to the Internet, to an intranet or to other audio systems in order to permit selective data exchange with external systems. Specific details of these requirements can be adapted to the individual needs.

Audio systems according to the invention can be composed of different components. Alternatives, however, also provide that audio systems according to the invention are formed in a single housing as an individual, self-contained device. Thus, even the multi-path loudspeaker and/or the data source for providing the input audio signal to be amplified can be integrated into the same housing in which the entire audio system is located. However, the at least one multi-path loudspeaker can also be arranged in its own separate housing, for example. In addition to the at least one multi-path loudspeaker, the audio system can also have further output options via headphones, structure-borne sound transducers and additional loudspeakers and/or devices. Suitable interfaces are known per se in the prior art.

It is also possible for the audio system to be equipped to determine and take into account the room acoustics. The measurement of the room acoustics is known per se in the prior art and can be integrated into audio systems according to the invention in this way.

In addition to the audio system itself, the invention also relates to a method according to the invention for operating such an audio system. This provides that the audio system, when the output audio signal is reproduced, the at least two distinct frequency band speakers are each only actuated in their respective useful frequency bands. In the hearing test mode, audio systems according to the invention can also use the multi-path loudspeaker for performing the at least one frequency-dependent hearing test. In this context, in a method for operating an audio system according to the invention, it can be provided that when the at least one, frequency-dependent hearing test is performed with the listener, the audio system emits hearing test audio signals via the at least two distinct frequency band speakers, wherein the respective hearing test audio signal is emitted via the frequency band speaker having the useful frequency band in which this respective hearing test audio signal is located. However, it is also possible to use a different loudspeaker, e.g. a single-path loudspeaker, to perform the frequency-dependent hearing test in the hearing test mode. For example, headphones or a bone-conducting receiver can be used as single-path loudspeakers to perform the frequency-dependent hearing test. The use of headphones is particularly recommended for conducting frequency-dependent hearing tests, especially when independent frequency-dependent hearing tests are to be performed with the left and right ears of the listener. In this case also, the result of the respective frequency-dependent hearing test can then be used according to the invention in the reproduction of the amplified output audio signals via the multi-path loudspeaker of the audio system according to the invention.

In the case of audio systems according to the invention, it is advantageously provided that a separate amplifier with its own level controller is connected upstream of each frequency band speaker in the audio system. It is particularly preferable in this case that a separate digital/analog converter is connected upstream of each level controller, wherein the digital/analog converters can be actuated by the digital signal processor. The respective digital/analog converters, level controllers and amplifiers can be designed as separate components in the signal processing unit of the audio system according to the invention and connected in series. However, it is also conceivable to use so-called controllable digital amplifiers with digital input, which in themselves have the functionality of a digital/analog converter, a level controller and also an amplifier and thus actuate the respective frequency band speaker directly as an integrated embodiment of these components. The particular amplifier can be a pure analog amplifier. However, it can also be a digital amplifier, which captures and amplifies an analog audio signal in order to deliver it as an amplified signal to the frequency band speaker. It could also be a so-called hybrid amplifier, which combines analog and digital amplification technology.

Audio systems according to the invention can be configured such that the digital signal processor actuates the respective frequency band speaker via the respective separate digital/analog converter, the respective separate level controller and the respective separate amplifier only in the useful frequency band of the respective frequency band speaker. In the context of the method according to the invention, it is therefore provided that the respective frequency band speaker is actuated by the digital signal processor via the respective separate digital/analog converter, the respective separate level controller and the respective separate amplifier only in the useful frequency band of the respective frequency band speaker.

Advantageous variants of audio systems according to the invention also provide that the audio system has a master processor for actuating the digital signal processor and the level controller. Such audio systems can be configured in such a way that the master processor actuates the respective amplifiers via the respective level controllers by means of a uniform level in the respective useful frequency band of the respective frequency band speaker. In the context of the method for operating the audio system it is thus provided that the respective amplifiers are actuated by the master processor via the respective level controllers by means of a uniform level in the respective useful frequency band of the respective frequency band speaker. It is also advantageous in this context if the master processor and/or the digital signal processor determines the uniform level in the respective useful frequency band as a function of the stored result of the frequency-dependent hearing test in the respective useful frequency band. The uniform level in the respective useful frequency band can be determined, for example, as a mean value of the gain function in this frequency band determined from the hearing test. In all cases, advantageous variants of the invention provide that a coarse adjustment is carried out via the amplifiers and level controllers connected upstream of the respective frequency band speaker and a fine adjustment is carried out via the upstream digital signal processor, wherein the gain function is obtained from the combination of the coarse adjustment and fine adjustment.

### BRIEF DESCRIPTION OF THE DRAWINGS

Further features and details of preferred embodiments of the invention are explained below by reference to exemplary embodiments according to the invention. In the drawings:

FIG. 1 shows a first schematic illustration of an exemplary embodiment according to the invention of an audio system;

FIG. 2 shows an illustration in which the components from FIG. 1 are illustrated in more detail;

FIG. 3 shows an example of a measured frequency-dependent hearing loss curve;

FIG. 4 shows an illustration of a frequency-dependent gain function determined from the hearing loss curve according to FIG. 3 and a coarse adjustment determined therefrom;

FIG. 5 shows the fine adjustment applied to the gain function and to the coarse adjustment from FIG. 4;

FIG. 6 shows an illustration with two different frequency-dependent hearing loss curves;

FIG. 7 shows the frequency-dependent gain functions determined from the hearing loss curves according to FIG. 6 and a gain function averaged therefrom;

FIG. 8 shows an extended form of the audio system of FIG. 1;

FIG. 9 shows an example of an audio system according to the invention, which can be used in conjunction with a home cinema;

FIG. 10 shows an audio system according to the invention integrated into a vehicle;

FIGS. 11 and 12 show audio systems according to the invention for supplying different zones in a house.

### DETAILED DESCRIPTION

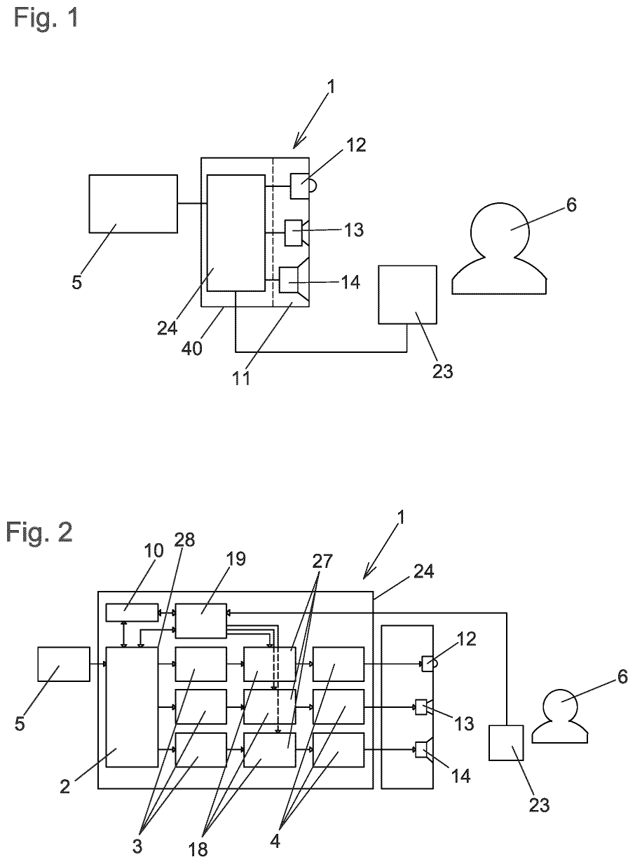

FIG. 1 shows a highly schematic diagram of an audio system 1 according to the invention. In this exemplary embodiment, the signal processing unit 24 is connected to an external data source 5. The audio system 1 comprises according to the invention a multi-path loudspeaker 11. In this exemplary embodiment, the latter comprises three frequency band speakers 12, 13 and 14. The frequency band speaker 12 is a tweeter, the frequency band speaker 13 is a midrange speaker, and the frequency band speaker 14 is a woofer. The frequency band speakers 12, 13 and 14 according to the invention thus have distinct useful frequency bands 15, 16 and 17. The multi-path loudspeaker 11 may be directly integrated into a housing 40 of the audio system 1, in which the signal processing unit 24 is also located. The dashed line in FIG. 1, however, indicates that the multi-path loudspeaker 11 can also be arranged in a separate housing, i.e. detached from the housing 40 of the audio system 1, as shown in FIG. 2.

As already explained, the data source 5 could of course also be an internal data source, which is integrated into the signal processing unit 24. A plurality of different data sources 5 can also be connected to or integrated into the signal processing unit 24.

For performing the frequency dependent hearing test in the hearing test mode, the audio system 1 according to FIG. 1 has an input unit 23 that can be operated by the listener 6. This input unit 23, as explained earlier, can be connected or connectable to the audio system 1 wirelessly but also by wires. It is equally possible to integrate the input unit 23 directly into the housing 40 of the audio system 1. It has already been explained above that there are a wide range of options for the input unit 23. Reference is therefore made to those comments here.

In FIG. 2, the structure of the signal processing unit 24 of the audio system 1 from FIG. 1 is now shown in more detail. The signal processing unit 24 comprises a digital signal processor 2, a plurality of digital/analog converters 3, a plurality of level controllers 18 and a plurality of amplifiers 4. It is easily discerned that in this exemplary embodiment also, a separate amplifier 4 with a separate level controller 18 is connected upstream of each frequency band speaker 12, 13 and 14. In any case, a separate digital/analog converter 3 is then connected upstream of the respective level controller 18. The digital/analog converters 3 are actuated by the digital signal processor 2. It is thus possible that the digital signal processor 2 actuates each frequency band speaker 12, 13 and 14 via a respective separate digital/analog converter 3, a respective separate level controller 18 and a respective separate amplifier 4 only in the useful frequency band 15, 16, 17 of the respective frequency band speaker 12, 13 and 14. The digital signal processor 2 thus ensures that each of the frequency band speakers 12, 13 and 14 is actuated only in the frequency band which corresponds to the user frequency band 15, 16 and 17 specified by its design. In order to actuate the signal processor 2 and the level controllers 18, the audio system 1 or its signal processing unit 24 preferably has a master processor 19, also realized in this exemplary embodiment. This master processor 19 controls the respective level controllers 18 of the respective amplifiers 4 advantageously by means of a uniform level 20, 21 and 22 in the respective useful frequency band 15, 16, 17 of the respective frequency band speaker 12, 13, 14. This will be explained in more detail later with reference to FIG. 4. In addition, the audio system 1 or its signal processing unit 24 also has a data storage device 10. This allows the results of the one or more frequency-dependent hearing tests performed with the audio system 1 to be recorded. The components of the signal processing unit 24 shown individually in FIG. 2 can be implemented as shown schematically here. However, it is also possible to combine different components together to form an integrated component. For example, the digital signal processor 2 and the master processor 19 could be combined into a single processor. This could also perform the storage function of the data storage device 10. Also, the external data source 5 could, as already described, be integrated into the signal processing unit 24, or into the master processor 19 or the digital signal processor 2. In addition, it should also be noted that the chain consisting of digital/analog converter 3, level controller 18 and amplifier 4 leading to any one of the frequency band speakers 12, 13 and 14 from the digital signal processor 2 could be implemented as one component in each case. The respective chain of digital/analog converter 3, level controller 18 and amplifier 4 could also be designed as a suitably controllable digital amplifier with digital input, which performs the functionalities of the digital/analog converter 3, the level controller 18 and the amplifier 4 in one. Such controllable digital amplifiers with digital input would then be controlled accordingly by the master processor 19.

The amplifiers 4 can be pure analog amplifiers or digital amplifiers, which output a corresponding analog signal to the respective frequency band speaker 12, 13 and 14.

FIGS. 1 and 2 also illustrate the option of designing audio systems according to the invention 1 as stand-alone, functioning systems in themselves. Thus, for example, the signal processing unit 24 and its components shown in FIG. 2 can be integrated into a single, common housing 40. In addition, the multi-path loudspeaker 11, the data source 5 and, if desired, also the input unit 23 can be integrated into this housing 40 of the audio system 1. Due to the data storage device 10, such audio systems 1 can be operated completely independently in both reproduction mode and in hearing test mode without any connection to the Internet or an intranet or to other networks. However, this does not of course exclude the possibility that audio systems 1 according to the invention can also communicate via corresponding networks for targeted data exchange with other audio systems 1 or other devices such as televisions, cinema systems.

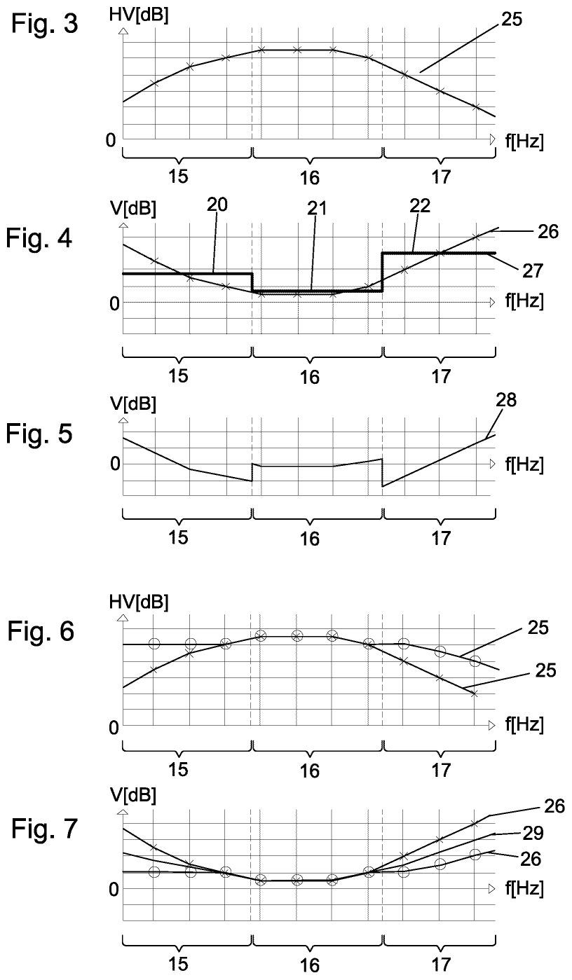

FIG. 3 now shows an example of a hearing loss curve 25, as was determined in the hearing test operating mode of the audio system 1 according to FIGS. 1 and 2 in the course of a frequency-dependent hearing test with the listener 6. In FIG. 3, the hearing loss HV in decibels is plotted against the frequency fin Hertz. The crosses show the measurement points, i.e. the measuring frequencies, at which the hearing threshold, i.e. the lower limit of hearing capacity, was actually determined in the course of the frequency-dependent hearing test. For this purpose, the audio system 1 according to FIGS. 1 and 2 emits an initially very quiet audio signal that becomes increasingly louder at each of these measurement frequencies via the multi-path loudspeaker 11 or, as explained further below, via headphones 30 or bone-conducting receivers 31 or the like. As soon as the listener 6 hears the signal, he/she outputs a corresponding signal to the signal processing unit 24 via the input unit 23. Each value marked in FIG. 3 with a cross is stored in the data storage device 10, the same operation is then carried out with the other measurement frequencies, so that in total the sequence of measurement points shown by the crosses in FIG. 3 is obtained. From this, the hearing loss curve 25 can then be generated via appropriate interpolation between the measurement points and stored as such by the audio system 1 in the data storage device 10 as the result of the frequency-dependent hearing test. From this frequency-dependent hearing loss curve 25, the exemplary frequency-dependent gain function 26 shown in FIG. 4, which is intended to compensate for the hearing losses contained in the hearing loss curve 25 individually recorded for the respective person 6, is then calculated in the digital signal processor 2. The frequency-dependent gain function 26 could also be stored in the data storage device 10 as the result of the frequency-dependent hearing test.

In FIGS. 3 and 4, the useful frequency bands 15, 16 and 17 of the frequency band speakers 12, 13 and 14 used in the multi-path loudspeaker 11 of the audio system 1 have already been drawn in. In the example shown here, the tweeter 12 has the useful frequency band 17, the midrange speaker 13 the useful frequency band 16 and the woofer 14 the useful frequency band 15. The master processor 19 or the digital signal processor 2 then calculate for each respective useful frequency band 15, 16 and 17 a uniform level 20, 21 and 22 in the respective useful frequency band 15, 16 and 17 depending on the stored result of the frequency-dependent hearing test. Preferably, in order to determine the levels 20, 21 and 22, the sections of the gain function 26 in the respective useful frequency band 15, 16 and 17 are averaged in each case, as shown in FIG. 4. For the calculation of the levels 20, 21 and 22 in the useful frequency bands 15, 16 and 17 on the basis of the gain function 26, however, other calculation rules can also be used instead of averaging. From the concatenation of the levels 20, 21 and 22, the coarse adjustment 27 is obtained in all cases. This coarse adjustment 27 is carried out with reference to FIG. 2 in the analog domain, by the master processor 19 actuating the respective level controllers 18 with the respective level 20, 21 and 22, so that the amplifiers 4 perform the corresponding level amplification in the respective useful frequency band 15, 16 and 17. In contrast, the fine adjustment 28 is carried out in the digital domain using the digital signal processor 2. FIG. 5 shows an example of the fine adjustment curve 28. FIGS. 4 and 5 show the respective gain V in decibels as a function of the frequency fin Hertz. The sum of the analog coarse adjustment 27 and the digitally performed fine adjustment 28 results in the gain function 26 required for compensating the hearing loss curve 25, as shown in FIG. 4.

For the sake of completeness, it is pointed out that FIGS. 3 to 5 of course relate to only a single example, which is intended to illustrate the mode of operation of the audio system 1. The procedure described thus serves only as an example to illustrate the principle, which can be realized within the scope of the invention in numerous different embodiments. For ease of comprehension, in FIGS. 4 and 5 the rising and falling slopes of the amplifiers 4 and the level controllers 18, which are usually present in practice at the edges of the useful frequency bands 15, 16 and 17 due to the design, have not been shown. However, these do not change the overall concept of amplification in audio systems according to the invention 1.

FIG. 6 shows now, by way of example, that with audio systems 1 according to the invention it is possible to work not only with a single hearing loss curve 25 or with a single frequency-dependent hearing test, but also with multiple hearing loss curves 25. In FIG. 6, the hearing loss HV in decibels is again plotted against the frequency f in Hertz. It is easy to see that the hearing loss curves 25 are identical in the medium frequency ranges, but differ significantly in the lower and higher frequencies. For example, these may be hearing loss curves 25 that were recorded with different people 6, 7, 8, 9. They could also be hearing loss curves 25 of a single person, where, for example, the hearing loss curve 25 marked by the crosses was recorded with the person's left ear and the hearing loss curve 25 marked with the circles was recorded with the person's right ear.

FIG. 7 now shows firstly the gain functions 26 calculated from the two hearing loss curves 25 of FIG. 6, as well as an averaged gain function 29 calculated by averaging these two gain functions 26. In FIG. 7, the gain Vin decibels is again plotted against the frequency fin Hertz. Such averaging operations can be used, for example, to calculate a mean gain function 29 for different hearing losses on the right and left ears of a single person 6, 7, 8, 9. Averaged gain functions 29 can also be used when the objective is to use the audio system 1 to achieve an averaged form of the gain for persons 6, 7, 8, 9 with different types of hearing loss. The division into coarse adjustment 27 and fine adjustment 28 and the amplification of the input audio signals based on it is carried out for averaged amplification functions 29 in the same manner as is explained by way of example with reference to FIGS. 4 and 5 for the gain function 26 shown there.

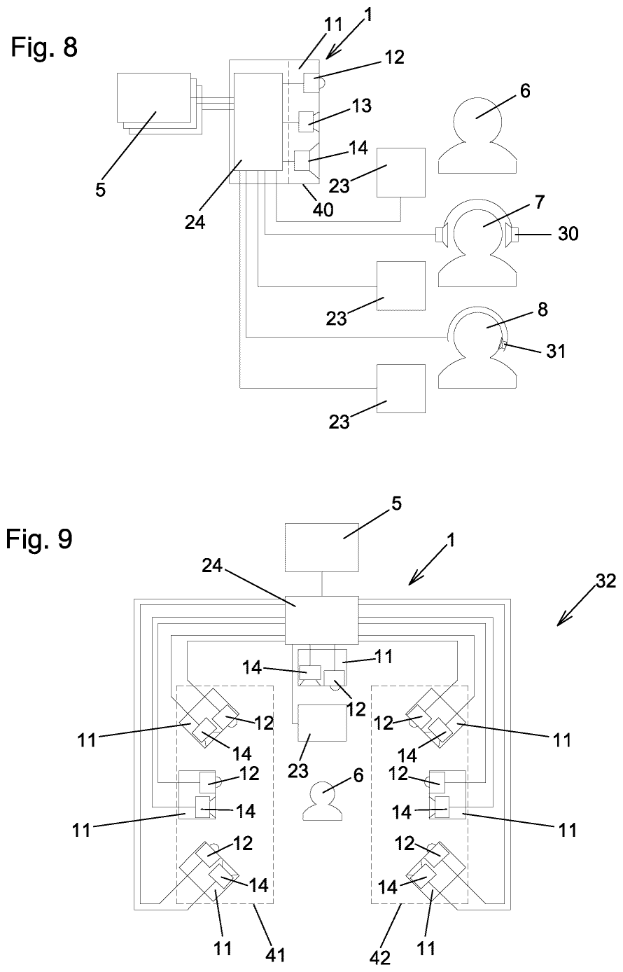

The audio system 1 according to the invention which is shown in FIG. 8 is an extension of the audio system 1 from FIG. 1. Reference is therefore made initially to the description of FIG. 1. FIG. 8 now illustrates by way of example that the audio system 1 can also be connected or connectable to a plurality of data sources 5 for providing input audio signals to be amplified. Of course, in deviation from the representation chosen here in FIG. 8, multiple such data sources 5 can also be integrated into the signal processing unit 24.

In addition, FIG. 8 also illustrates the fact that the audio system 1 can comprise a plurality of input units 23 and also different output units for reproducing amplified output audio signals. Thus, in FIG. 8, in addition to the multi-path loudspeaker 11 which is also already present in FIG. 1, a pair of headphones 30 and a bone-conducting receiver 31 are provided, with which the additional persons 7 and 8 can be supplied. Both the headphones 30 and the bone-conducting receiver 31 can be used for both the hearing test mode and the reproduction mode. Thus, it is possible to perform the frequency-dependent hearing test both via the multi-path loudspeaker 11 as well as via the headphones 30 and also via the bone-conducting receiver 31. If, as shown in FIG. 8, multiple persons 6, 7 and 8 are to be supplied with output audio signals, then for each person 6, 7 and 8 a separate frequency-dependent hearing test can be performed with the audio system 1 and stored in the data storage device 10. It is also possible, particularly with the headphones 30, to perform a hearing test on one person, here e.g. the person 7, separately for the left ear and the right ear, in order to then store this in the data storage device 10 and to use it as a basis for the amplification for reproducing the amplified output audio signals in the reproduction mode. The performance of the frequency-dependent hearing tests and the calculation of the gain function 26 from the hearing loss curve 25 is carried out in each case as described above, wherein the reproduction of the amplified output audio signal can take place in the form according to the invention, e.g. via the multi-path loudspeaker 11. The gain functions 26 can be averaged, as illustrated by way of FIGS. 6 and 7. For example, to generate an averaged gain function for different individuals 6, 7 and/or 8 or an averaged gain function 29 for a person 6, 7 and/or 8, for whom separate frequency-dependent hearing tests were performed with the right and left ear.

In the reproduction mode, amplified output audio signals can be reproduced according to the invention via the multi-path loudspeaker 11, and via the headphones 30 and the bone-conducting receiver 31 the reproduction of the amplified output audio signals can initially also be carried out according to the prior art. Here, however, a modification according to the invention is also possible, for example, by the persons 7 and 8 receiving the amplified output audio signals via the frequency band speakers 12, 13, 14 of the multi-path loudspeaker 11 and additionally also selectively amplified in the useful frequency band of the headphones 30 and/or the bone-conducting receiver 31. The headphones 30 and/or the bone-conducting receiver 31 can then be used as an additional frequency band speaker of the multi-path loudspeaker 11, so to speak. For example, it would be possible to selectively amplify the output audio signals in higher useful frequency bands for each person 7 and 8 via the headphones 30 and/or the bone-conducting receiver 31 and to supply these persons 7 and 8 with the low frequencies or with structure-borne sound additionally via the multi-path loudspeaker 11 and in particular the frequency band speaker 14 designed as a woofer. In the same way, it can also be an option to supply the structure-borne sound, for example, via a piece of seating or reclining furniture with an integrated structure-borne sound transducer or structure-borne sound speaker.

FIG. 9 shows an audio system 1 for a home cinema 32 in a schematic representation. The screen and projector used to reproduce the images are not shown here. In the example shown in FIG. 9, the audio system 1 has a total of seven multi-path loudspeakers 11 each with two frequency band speakers 12 and 14, thus each having a tweeter and a woofer. Three of the multi-path loudspeakers 11 are arranged to the left of the person 6. Three additional multi-path loudspeakers 11 are located on the right-hand side of the person 6. In addition, there is a seventh multi-path loudspeaker 11 located centrally in front of the person 6. In this configuration, it is now possible, for example, that the person 6 carries out a frequency-dependent hearing test successively using the input unit 23 with each individual multi-path loudspeaker 11 and that this total of seven frequency-dependent hearing tests are stored in the data storage device 10 of the audio system 1, in order thus to generate a separate gain function 26 with corresponding coarse adjustment 27 and fine adjustment 28 for each of these multi-path loudspeakers 11 in the manner described in principle above, so that the reproduction of the amplified output audio signals takes place via each of the seven multi-path loudspeakers 11, taking into account the stored amplification function 26 in each case. By way of deviation from this, it would of course also be possible to group the multi-path loudspeakers 11 together in order to actuate the respective group of multi-path loudspeakers using the same gain function 26. For example, FIG. 9 shows a group 41 on the left with three multi-path loudspeakers 11 and a group on the right 42 also with three multi-path loudspeakers 11. The seventh multi-path loudspeaker 11, which is centrally positioned in front of the person 6, could be actuated e.g. with an averaged gain function 29, but also with its own gain function 26. Here there are numerous possibilities to adapt the system shown in FIG. 9 accordingly. The structure and mode of operation of the signal processing unit 24 of the audio system 1 from FIG. 9 correspond in principle to that described in FIG. 2, but with a corresponding number of digital/analog converters 3, level controllers 18 and amplifiers 4, so that each of the frequency band speakers 12 and 14 of the multi-path loudspeakers 11 can be individually actuated accordingly in its respective useful frequency band.

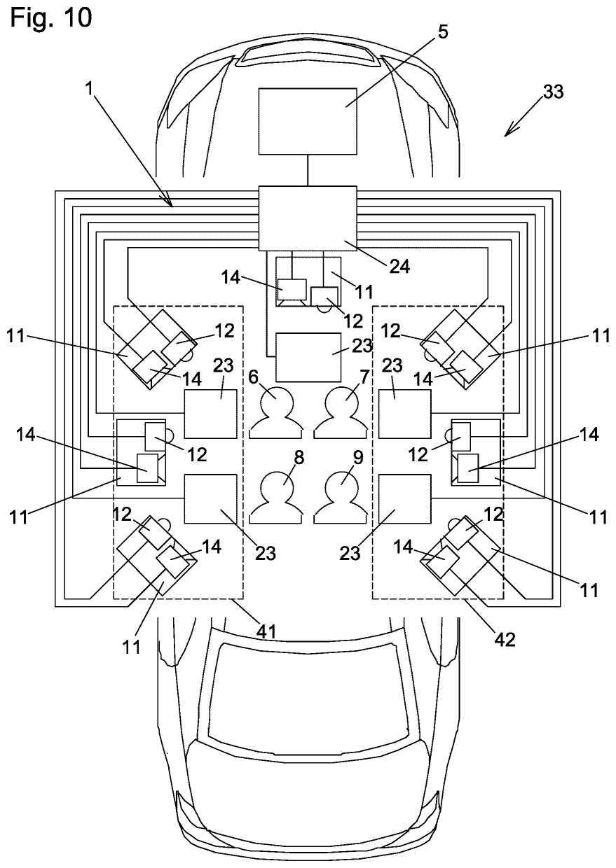

FIG. 10 now shows, by way of example, a configuration according to the invention of an audio system 1, such as can be used in a vehicle 33. The structure of the signal processing unit 24 and its mode of operation again correspond in principle to the variants described with reference to FIGS. 2 to 7 in a corresponding duplication of the components. The structure of this audio system 1 in FIG. 10 is similar to the structure of the audio system 1 in FIG. 9, except that here, as an example, a total of four persons 6, 7, 8 and 9 are located in the space to be sonicated, i.e. in the vehicle 33 in which the reproduction of amplified output audio signals takes place. In contrast to FIG. 9, here a plurality of input units 23 are present, so that each person 6, 7, 8 and 9 can perform one or more separate frequency-dependent hearing tests and the resulting gain functions 26 with their coarse adjustment 27 and fine adjustment 28 or suitably averaged gain functions 29 can also be used with coarse adjustment 27 and fine adjustment 28 for the amplification in the audio system 1. Many different variants are conceivable here. A simple variant is, for example, that only the driver 6 of the vehicle 33 performs a frequency-dependent hearing test and all multi-path loudspeakers 11 in the vehicle 33 are actuated with the same gain function 26. However, it is also possible to carry out at least one frequency-dependent hearing test with all persons 6, 7, 8 and 9, and to generate from this an averaged gain function 29 with coarse adjustment 27 and fine adjustment 28 for all multi-path loudspeakers 11. It is equally possible to actuate, for each of the persons 6, 7, 8 and 9, only the multi-path loudspeakers 11 in the immediate vicinity with the corresponding gain function 26 generated for this person 6, 7, 8, 9 on the basis of at least one corresponding hearing test. Again, the multi-path loudspeakers 11 can be grouped together, e.g. also into a group on the left 41 and a group on the right 42.

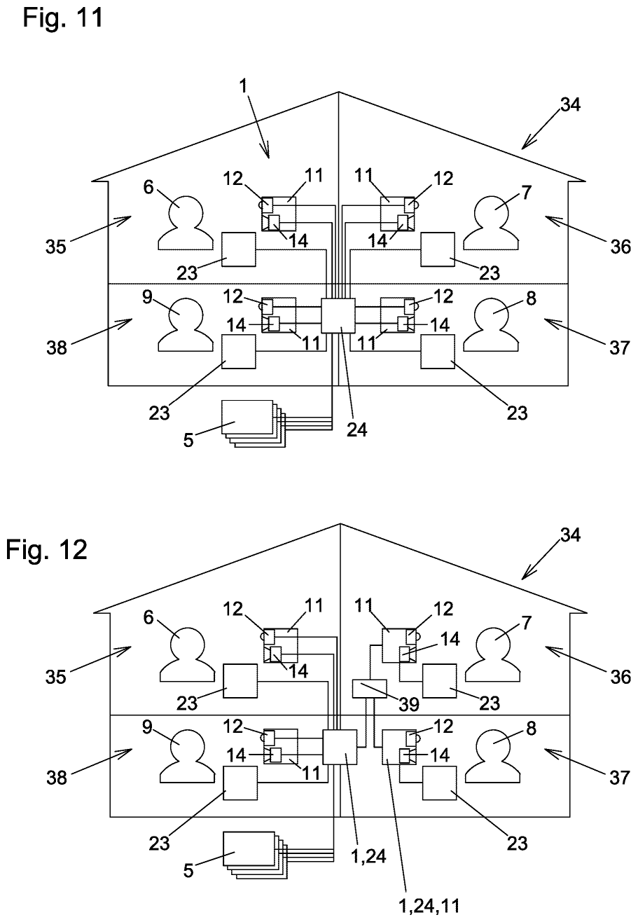

FIG. 11 shows a schematized variant of an audio system 1 according to the invention, in which a central signal processing unit 24 of the audio system 1 is used to output suitably amplified output audio signals via different multi-path loudspeakers 11 in different rooms 35, 36, 37 and 38. The person 6, 7, 8 or 9 present in the room 35, 36, 37 or 38 can carry out a frequency-dependent hearing test via the respective input unit 23 present there with the multi-path loudspeaker 11 provided, so that the result of the latter can be stored in the data storage device 10 in the manner described above and can be retrieved for the corresponding generation of the output audio signal in this room 35, 36, 37 or 38. Again, a plurality of data sources 5 can be used to feed different input audio signals into the audio system 1 and amplify them there accordingly. The structure and mode of operation of the signal processing unit 24 corresponds again, with an appropriate duplication of the components, to the procedure described above with reference to FIGS. 1 to 7. Here, also, it is of course possible instead to work with averaged gain functions 29 and/or to amplify the output audio signals for different rooms 35, 36, 37 and 38 in the same manner. Here, also, there are, of course, various possibilities for applying the principle according to the invention.

FIG. 12 is a variation of FIG. 11. This also involves a multi-room variant for different rooms 35, 36, 37 and 38. In this application according to the invention as a decentralized audio system 1 for a multi-room 34 configuration, two or more audio systems 1 logged into a network 39 can exchange the respectively stored frequency-dependent hearing tests of one or more persons 6, 7, 8 and 9 with one another, preferably without access to external databases, to determine the frequency-dependent gain functions 26 or suitably averaged gain functions 29 and the resulting coarse adjustment 27 and fine adjustment 28 for multiple rooms 35, 36, 37 or 38 and audio systems 1 in each case. For example, an additional supplemented audio system 1 can use the parameters of a pre-existing audio system 1, if required. The network 39 can be a wired or wireless network, such as a LAN, WLAN, or Bluetooth link.

List of Reference Signs:

| | |
|---|---|
| 1 | audio system |
| 2 | digital signal processor |
| 3 | digital/analog converter |
| 4 | amplifier |
| 5 | data source |
| 6 | person |
| 7 | person |
| 8 | person |
| 9 | person |
| 10 | data storage device |
| 11 | multi-path loudspeaker |
| 12 | frequency band speaker |
| 13 | frequency band speaker |
| 14 | frequency band speaker |
| 15 | useful frequency band |
| 16 | useful frequency band |
| 17 | useful frequency band |
| 18 | level controller |
| 19 | master processor |
| 20 | level |
| 21 | level |
| 22 | level |
| 23 | input unit |
| 24 | signal processing unit |
| 25 | hearing loss curve |
| 26 | gain function |
| 27 | coarse adjustment |
| 28 | fine adjustment |
| 29 | averaged gain function |
| 30 | headphones |
| 31 | bone-conducting receiver |
| 32 | home cinema |
| 33 | vehicle |
| 34 | multi-room |
| 35 | room |
| 36 | room |
| 37 | room |
| 38 | room |
| 39 | network |
| 40 | housing |
| 41 | left group |
| 42 | right group |

1. An audio system for reproducing amplified output audio signals by at least one loudspeaker of the audio system, the audio system comprising:
a signal processing unit for actuating the loudspeaker, the signal processing unit comprising a digital signal processor, at least one digital/analog converter and at least one amplifier;
the signal processing unit is connected or connectable to a data source for providing input audio signals to be amplified, or the data source is integrated into the signal processing unit;
a reproduction mode for reproducing the amplified output audio signals and a hearing test mode for carrying out at least one frequency-dependent hearing test with a listener
a data storage device for storing a result of the at least one, frequency-dependent hearing test carried out with the audio system;
wherein the loudspeaker is a multi-path loudspeaker having at least two distinct frequency band speakers for reproducing the amplified output audio signal, with the at least two distinct frequency band speakers each having distinct useful frequency bands.

2. The audio system as claimed in claim 1, a wherein the at least one amplifier includes a separate amplifier (4) with a separate level controller connected upstream of each of the frequency band speakers.

3. The audio system as claimed in claim 2, wherein the at least one digital/analog converter includes a separate digital/analog converter connected upstream of each of the separate level controllers, and the digital/analog converters are actuatable by the digital signal processor.

4. The audio system as claimed in claim 3, wherein the digital signal processor is configured to actuate the respective frequency band speaker via the respective separate digital/analog converter, the respective separate level controller and the respective separate amplifier only in the useful frequency band of the respective frequency band speaker.

5. The audio system as claimed in claim 2, further comprising a master processor configured to actuate the digital signal processor and the level controller.

6. The audio system as claimed in claim 5 the master processor actuates the respective separate amplifiers via the respective level controllers by a uniform level in the respective useful frequency band of the respective frequency band speaker.

7. The audio system as claimed in claim 6, wherein at least one of the master processor or the digital signal processor determines the uniform level in the respective useful frequency band as a function of the stored result of the frequency-dependent hearing test in the respective useful frequency band.

8. The audio system as claimed in claim 1, wherein for carrying out the frequency-dependent hearing test the audio system is connected or connectable to an input unit which is operable by the listener, or is integrated into the audio system.

9. A method for operating the audio system as claimed in claim 1, the method comprising the audio system actuating, when reproducing the output audio signal, each of the at least two distinct frequency band speakers only in the respective useful frequency bands.

10. The method as claimed in claim 9, further comprising the audio system emitting, when the at least one frequency-dependent hearing test is performed with the listener, hearing test audio signals via the at least two distinct frequency band speakers, wherein the respective hearing test audio signal is emitted via the frequency band speaker having the useful frequency band in which this respective hearing test audio signal is located.
